(19) United States
(12) Patent Application Publication
(10) Pub. No.: US 2018/0110646 A1
(43) Pub. Date: Apr. 26, 2018

(54) Female Urination Assembly

(71) Applicants: Tex Brooks, Henderson, NV (US); Nancy Kane, Henderson, NV (US)

(72) Inventors: Tex Brooks, Henderson, NV (US); Nancy Kane, Henderson, NV (US)

(21) Appl. No.: 15/335,048

(22) Filed: Oct. 26, 2016

(51) Int. Cl. A61F 5/455

(52) U.S. Cl. CPC A61F 5/4556; A61F 2005/4402

(57) ABSTRACT

A female urination assembly for facilitating a female to urinate while standing up includes a cone. The cone is selectively aligned with female genitalia thereby facilitating the cone to direct a flow of urine in a selected direction. A coating is positioned on the cone. The coating is comprised of a resinous material such that the coating facilitates structural rigidity to the cone.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98.

The disclosure and prior art relates to urination devices and more particularly pertains to a new urination device for facilitating a female to urinate while standing up.

### BRIEF SUMMARY OF THE INVENTION

An embodiment of the disclosure meets the needs presented above by generally comprising a cone. The cone is selectively aligned with female genitalia thereby facilitating the cone to direct a flow of urine in a selected direction. A coating is positioned on the cone. The coating is comprised of a resinous material such that the coating facilitates structural rigidity to the cone.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a female urination assembly according to an embodiment of the disclosure.

FIG. 2 is a bottom view of an embodiment of the disclosure.

FIG. 3 is a front view of an embodiment of the disclosure.

FIG. 4 is a perspective in-use view of an embodiment of the disclosure.

FIG. 5 is a cross sectional view taken along line 5-5 of FIG. 3 of an embodiment of the disclosure.

FIG. 6 is a front perspective view of a dispenser of an embodiment of the disclosure.

### DETAILED DESCRIPTION OF THE INVENTION

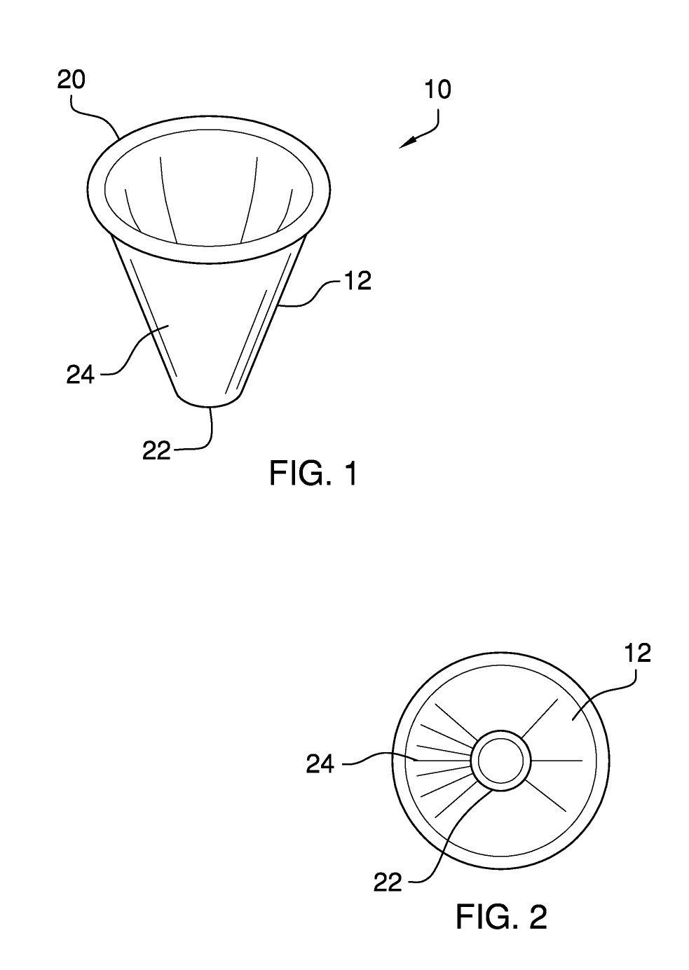

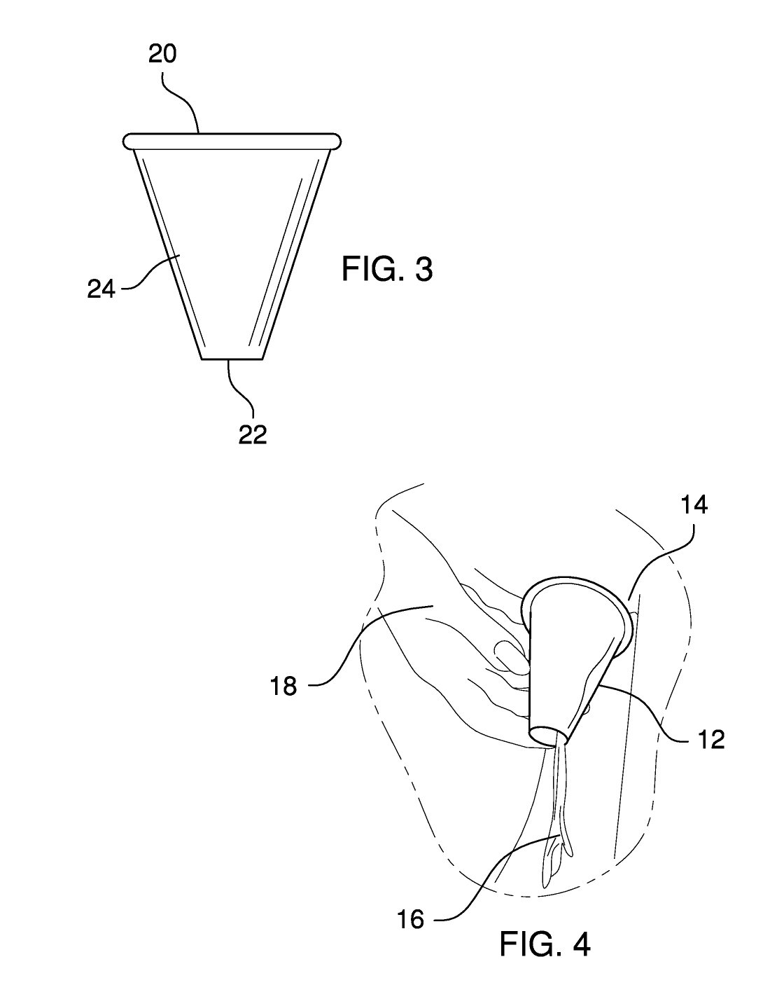

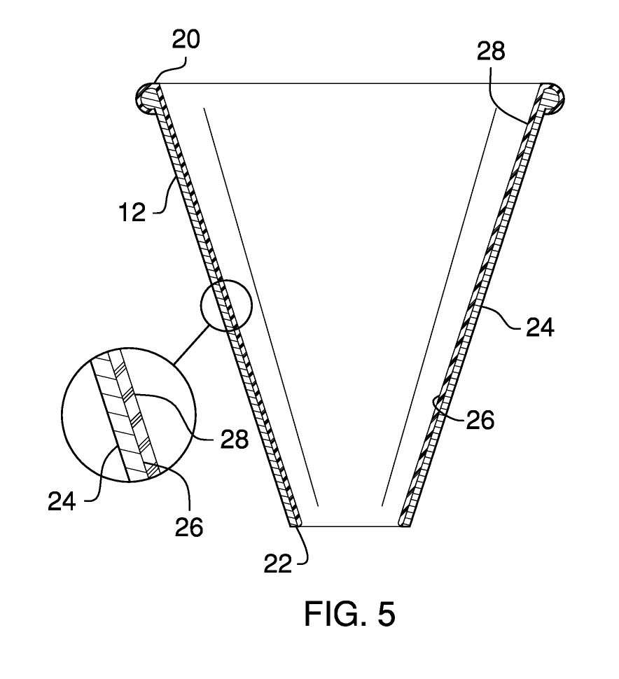

With reference now to the drawings, and in particular to FIGS. 1 through 6 thereof, a new urination device embodying the principles and concepts of an embodiment of the disclosure and generally designated by the reference numeral 10 will be described.

As best illustrated in FIGS. 1 through 6, the female urination assembly 10 generally comprises a cone 12 that may be aligned with female genitalia 14. Thus, the cone 12 directs a flow of urine 16 in a selected direction. The cone 12 is employed in a public restroom or the like. Thus, a female 18 may urinate without contacting a contaminated surface such as a public toilet seat or the like. The cone 12 enhances hygiene during female urination in the public restroom or the like.

The cone 12 has a first end 20, a second end 22 and an outer wall 24 extending therebetween. Each of the first end 20 and the second end 22 are open. The outer wall 24 tapers inwardly between the first end 20 and the second end 22. The first end 20 is selectively positioned against the female genitalia 14 thereby facilitating the first end 20 to capture the urine 16. The outer wall 24 has an inside surface 26.

The cone 12 is comprised of a fluid permeable material. Thus, the cone 12 degrades when the cone 12 is flushed down a toilet. The fluid impermeable material may be a non-woven fabric or the like. Thus, the non-woven fabric may disintegrate when the non-woven fabric is exposed to water.

A coating 28 is provided and the coating 28 is positioned on the cone 12. The coating 28 is comprised of a resinous material. Thus, the coating 28 imparts structural rigidity to the cone 12. The coating 28 is comprised of a fluidly resilient material. Thus, the coating 28 resists being dissolved by the urine 16. The coating 28 dissolves when the cone 12 is flushed down the toilet thereby facilitating the cone 12 to degrade. Moreover, the coating 28 is positioned on the inside surface 26 of the cone 12 and the coating 28 completely covers the inside surface 26.

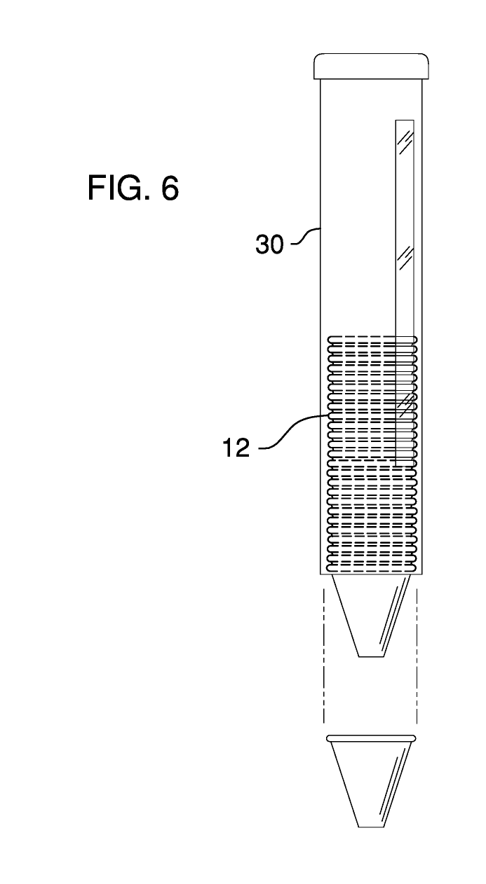

As shown in FIG. 6, a dispenser 30 may be provided and the dispenser 30 may be mounted to a wall or the like. The dispenser 30 may comprise a horizontally oriented cylinder or the like. A plurality of the cones 12 may be positioned within the dispenser 30. Each of the cones 12 may be selectively removed from the dispenser 30. Moreover, the dispenser 30 may be positioned in a public bathroom or the like.

In use, the cone 12 is positioned against the female's genitalia 14 when the female 18 is urinating in a public bathroom or the like. The first end 20 captures the urine 16 and the second end 22 directs the urine 16 into a toilet. Thus, the female 18 does not have to sit on an unclean or otherwise contaminated toilet. The coating 28 resists being dissolved by the urine 16. The cone 12 is flushed down the toilet when the female 18 is finished urinating. The coating 28 dissolves when the cone 12 is exposed to water for an extended period of time. Thus, the cone 12 degrades in the water.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A female urination assembly being configured to facilitate a female to urinate while standing, said assembly comprising:
a cone being configured to be aligned with female genitalia thereby facilitating said cone to direct a flow of urine in a selected direction, said cone having a first end, a second end and an outer wall extending therebetween, each of said first end and said second end being open, said cone having a circumferential bulge extending outwardly along said first end defining a lip around said first end; and
a coating being positioned on said cone, said coating being comprised of a resinous material such that said coating provides structural rigidity to said cone.

2. The assembly according to claim 1, wherein said outer wall tapers inwardly between said first end and said second end, said first end being configured to be positioned against the female genitalia thereby facilitating said first end to capture the urine, said outer wall having an inside surface.

3. The assembly according to claim 2, wherein said cone is comprised of a fluid permeable material wherein said cone is configured to degrade when said cone is flushed down a toilet.

4. (canceled)

5. A female urination assembly being configured to facilitate a female to urinate while standing, said assembly comprising:
a cone being configured to be aligned with female genitalia thereby facilitating said cone to direct a flow of urine in a selected direction, said cone having a first end, a second end and an outer wall extending therebetween, each of said first end and said second end being open, said cone having a circumferential bulge extending outwardly along said first end defining a lip around said first end, said outer wall tapering inwardly between said first end and said second end, said first end being configured to be positioned against the female genitalia thereby facilitating said first end to capture the urine, said outer wall having an inside surface, said cone being comprised of a fluid permeable material wherein said cone is configured to degrade when said cone is flushed down a toilet; and
a coating being positioned on said cone, said coating being comprised of a resinous material such that said coating facilitates structural rigidity to said cone, said coating being configured to dissolve when said cone is flushed down the toilet thereby facilitating said cone to degrade, said coating being positioned on said inside surface of said cone, said coating completely covering said inside surface.
